(19) United States
(12) Patent Application Publication
(10) Pub. No.: US 2026/0248908 A1
(43) Pub. Date: Aug. 27, 2026

(54) POTENTIATION OF AN INNATE IMMUNE RESPONSE IN MOLLUSKS

(71) Applicant: THE CAWTHRON INSTITUTE TRUST BOARD, Nelson (NZ)

(72) Inventors: Lizenn DELISLE, Nelson (NZ); Anne ROLTON, Nelson (NZ); Julien VIGNIER, Nelson (NZ)

(21) Appl. No.: 19/189,940

(22) Filed: Apr. 25, 2025

(30) Foreign Application Priority Data
Apr. 26, 2024 (NZ) 810485

(51) Int. Cl. A61K 39/245; A61K 39/00; A61K 39/002; A61K 39/02; A61P 37/04

(52) U.S. Cl. CPC A61K 39/245; A61K 39/002; A61K 39/107; A61P 37/04; A61K 2039/552

(57) ABSTRACT

A method of potentiating the immune response of a mollusk so that a population of the mollusk is less susceptible to disease on subsequent exposure to a pathogen is described. The method includes exposing the mollusk to an inactivated antigen preparation where the antigens are derived from the pathogen and inactivated by exposure to an ethylenimine.

### TECHNICAL FIELD

The invention relates to compositions and methods for use in reducing morbidity in populations of mollusks. In particular, although not exclusively, the invention relates to the reduction of morbidity in a population of farmed oysters where the morbidity is attributed to infection with a virus, e.g., Ostreid herpesvirus-1 (OsHV-1).

### BACKGROUND ART

The occurrence of mass mortality events and the emergence of infectious diseases affecting marine organisms have increased dramatically in recent years, exacerbated by a changing environment (Lafferty et al., 2004; Burge et al., 2014; Harvell et al., 2019). These outbreaks can have disastrous consequences on biodiversity and cause rapid population declines, particularly on cultured livestock (Daszak et al., 2000; Wiethoelter et al., 2015). Diseases are the major limiting factor for the expansion of the aquaculture industry, with losses attributed to infectious microbial diseases alone exceeding US$6 billion per annum (The International Bank for Reconstruction and Development, 2014). One striking example is Pacific oyster mortality syndrome (POMS), which is associated with the detection of the Ostreid Herpes virus-1 (OsHV-1) and its variants, and has decimated Pacific oysters, Crassostrea gigas, worldwide for the last 15 years (Barbosa-Solomieu et al., 2005; see for review EFSA, 2010, 2015; Pernet et al., 2016). The virus induces an immune-compromised state in infected oysters, which evolves towards subsequent bacteraemia by opportunistic bacterial pathogens, leading to mortality up to 100% in juveniles (De Lorgeril et al., 2018). Recorded for the first time in France in 2008, OsHV-1 μvar rapidly spread along the European coastline (EFSA, 2015; Pernet et al., 2016; Segarra et al., 2016) and closely related variants of the virus were further detected during mortality events in Australia (Jenkins et al., 2013), New Zealand (Keeling et al., 2014), Korea (Hwang et al., 2013) and more recently, California (Burge et al., 2021). The inability to contain the rapid spread of the virus combined with an absence of therapeutic treatments, resulted in huge losses of aquaculture stocks. Selective breeding to improve resistance to POMS (or OsHV-1) has shown potential as a prevention strategy, with moderate to high heritability for survival achieved during OsHV-1 infection (Dégremont et al., 2015; Camara et al., 2017; Divilov et al., 2019; Gutierrez et al., 2020).

Implementation of a breeding programme and access to selectively bred stocks can however be economically challenging for many end-users, prompting the need for new, accessible, and complementary mitigation strategies to reduce the impact of diseases.

Vaccination has proven to be an effective preventative measure for many major diseases affecting livestock, including fish and more recently, invertebrates such as crustaceans (Witteveldt et al., 2004; Dadar et al., 2016; Virol and Feng, 2017; Bøgwald and Dalmo, 2019; Yang et al., 2020).

Invertebrates are lacking a conventional adaptive immune system (i.e., lymphocytes or antibodies), instead relying on innate immunity to prevent the infection of invading pathogens (Buchmann, 2014; Netea et al., 2020). Numerous studies have reported that invertebrates also possess diverse forms of immune ‘memory’ in which a potentiated immune response (resulting in a reduction of host susceptibility to the infection) has been recorded following a secondary exposure to a pathogen (immune priming) (Netea et al., 2011; Milutinović and Kurtz, 2016; Sheehan et al., 2020; Yao et al., 2021). For instance, in the scallop Chlamys farreri, a first short exposure to the pathogen Vibrio anguillarum increased phagocytosis, acid phosphatase activity and survival following a second encounter with the pathogen (Cong et al., 2008). Pacific oysters stimulated primarily by heat-killed Vibrio splendidus also displayed stronger immune responses at cellular and molecular levels when they were subjected to a secondary challenge with the live bacteria (Zhang et al., 2014). More recently, Lafont et al. (2017, 2020) showed that injection of Polyinosinic: polycytidylic acid or Poly (I:C), a synthetic analog of double stranded RNA with immunostimulant properties, induced a long-lasting antiviral response in Pacific oysters, protecting them against subsequent OsHV-1 infection in nature. In bivalves, hemocytes play a central role in immunity, identifying and destroying pathogens through phagocytosis, oxidative stress, apoptosis and autophagy—functions that can be characterized using flow cytometry and molecular analyses (Cheng, 1996; Allam and Ford, 2006; Labreuche et al., 2006; Allam and Raftos, 2015; Buchmann and Holmes, 2015; Morga et al., 2017; Picot et al., 2022). Transcriptome analyses in oysters primed with Poly (I:C) have identified several pattern recognition receptors (PRRs) involved in antiviral signaling including; retinoic acid-inducible-gene-I and Toll-like-receptors homologs of the Jak-Stat pathway, stimulator of interferon genes, interferon regulatory factors and many IFN-stimulated genes (i.e. Viperin or ADAR) which are all implicated in detection of virus and antiviral function (Green and Montagnani, 2013; Green et al., 2015c, 2015a; Wang et al., 2018; Agius et al., 2020).

It is an object of the present invention to a method of potentiating the innate immune response in a mollusk. It is an object of the present invention to provide a composition for use in a method of potentiating the innate immune response in a mollusk. It is an object of the present invention to provide a method of preparing a composition for use in a method of potentiating the innate immune response in a mollusk. These objects are to be read in the alternative with each other and the object at least to provide a useful choice in the selection of such compositions or methods.

### SUMMARY OF INVENTION

In a first aspect a method of potentiating the innate immune response of a mollusk against a pathogen is provided. The method comprises contacting the mollusk with a composition comprising an inactivated pathogen-derived antigen where the pathogen-derived antigen has been inactivated by contacting with an ethylenimine. The method is advantageously used in the farming of mollusks where the mollusks may be contacted with the composition in the hatchery before transfer to the farm.

Preferably the mollusk is a bivalve mollusk. Preferably the bivalve mollusk is a species of mussel or oyster. When the bivalve mollusk is a species of mussel it is preferably Perna canaliculus (New Zealand Green-lipped Mussel). When the bivalve mollusk is a species of oyster it is preferably selected from the group consisting of: Crassostrea gigas (Pacific Oyster); Crassostrea sikamea (Kumamoto Oyster); Crassostrea virginica (American Oyster); Ostrea angasi (Australian Flat Oyster); Ostrea edulis (European Flat Oyster); Ostrea lurida (Olympia Oyster); and Tiostrea chilensis (Bluff Oyster). More preferably the species of oyster is Crassostrea gigas (Pacific Oyster) or Ostrea edulis (European Flat Oyster). Most preferably the mollusk is Crassostrea gigas (Pacific Oyster).

Preferably the pathogen is a bacterium, a parasitic protozoan, a parasitic rhizarian, or a virus. More preferably the pathogen is a virus. Most preferably the virus is Ostreid herpes virus-1 (OsHV-1). When the pathogen is a bacterium the pathogen is preferably a species of the genus Vibrio (Vibrio sp.). When the pathogen is a parasitic protozoan the pathogen is preferably Perkinsus olseni. When the pathogen is a parasitic rhizarian the pathogen is preferably a species of the genus Bonamia (Bonamia sp.). More preferably the species of the genus Bonamia is selected from the group consisting of: Bonamia exitiosa, Bonamia ostreae; and Bonamia roughleyi.

In a first embodiment of the first aspect the mollusk is Ostrea edulis and the pathogen is Bonamia ostreae. In a second embodiment of the first aspect the mollusk is Crassostrea gigas and the pathogen is Ostreid herpes virus-1 (OsHV-1). In a third embodiment of the first aspect the pathogen is Perkinsus olseni and the mollusk is Perna canaliculus. In a fourth embodiment of the first aspect the pathogen is Vibrio sp. and the mollusk is one or more species of mussel (e.g., Perna canaliculus) or oyster (e.g., Crassostrea gigas).

Inactivated pathogen-derived antigen may be prepared by ethylenimine (e.g., BEI) inactivation, formaldehyde inactivation, heat inactivation, and freeze-thaw cycling of the pathogen. Inactivated pathogen-derived antigen may also be prepared by nucleic acid or protein extraction of the pathogen. The inactivated pathogen-derived antigen is preferably prepared by ethylenimine inactivation. The inactivated pathogen-derived antigen is most preferably prepared by binary ethylenimine (BEI) inactivation. Multiple immune related genes are advantageously upregulated on exposure to BEI inactivated pathogen-derived antigen.

In a second aspect a composition for use in a method of potentiating the innate immune response of a mollusk is provided. The composition comprises an inactivated pathogen-derived antigen where the pathogen-derived antigen has been inactivated by contacting with an ethylenimine. Preferably, the composition is a dispersion of the inactivated pathogen-derived antigen in sterile seawater (SSW).

Preferably the antigen is derived from a pathogen that is a bacterium, a parasitic protozoan, a parasitic rhizarian, or a virus. In a first embodiment of the second aspect the pathogen is Vibrio sp. In a second embodiment of the second aspect the pathogen is Perkinsus olseni. In a third embodiment of the second aspect the pathogen is Bonamia ostreae. In a fourth embodiment of the second aspect the pathogen is Ostreid herpes virus-1 (OsHV-1).

The inactivated pathogen-derived antigen is advantageously prepared by ethylenimine inactivation. Multiple immune related genes are upregulated on exposure to binary ethylenimine (BEI) inactivated pathogen-derived antigen.

In a third aspect a method of preparing a composition for use in a method of potentiating the innate immune response of a mollusk is provided. The method comprises inactivating a pathogen-derived antigen by contacting with an ethylenimine. Preferably the ethylenimine is binary ethylenimine (BEI).

In the description and claims of this specification the following abbreviations, acronyms, terms and phrases have the meaning provided: “antigen” means a preparation that induces an immune response; “CAS RN” means Chemical Abstracts Service (CAS, Columbus, Ohio) Registry Number; “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “farm” means an area of land or water used for growing crops and rearing animals; “inactive” means incapable of causing an infection; “morbidity” means the condition of suffering from a disease; “moribund” means at the point of death; “pathogen-derived antigen” means any modified or unmodified antigenic material or substance obtained from a pathogen irrespective of its form; “potentiate” means increase the likelihood of an effective reaction or response; and “virion” means a viral particle. A paronym (derivative) of any of the defined terms has a corresponding meaning. E.g., “antigenic” is a paronym of the defined term “antigen”.

The terms “first”, “second”, “third”, etc. used with reference to elements, features, integers, or other limitations, of the matter described in the Summary of Invention, or when used with reference to alternative aspects or embodiments are not intended to imply any order of preference. Elements, features, integers, or other limitations, of the elements described in the Summary of Invention are identified in order of preference by the introductory “preferably . . . ”, “more preferably . . . ”, “yet more preferably . . . ” and so on. Preferred combinations of elements, features, integers, or other limitations, of the matter described in the Summary of Invention are similarly identified.

Where concentrations or ratios of reagents are specified the concentration or ratio specified is the initial concentration or ratio of the reagents. Similarly, where a pH or pH range is specified, the pH or pH range specified is the initial pH or pH range. Where values are expressed to one or more decimal places standard rounding applies. For example, 1.7 encompasses the range 1.650 recurring to 1.749 recurring.

Where a parameter is expressed as being “about” a specified range or value the term is used to indicate tolerance for some variation of the specified range or value (with the proviso that the parameter dependent effect is still achieved). In the absence of any other proviso the term “about” should be understood to indicate a tolerance of no greater than 5% above or below the upper and lower limits, respectively, of the specified range or plus or minus 5% of the specified value.

The invention will now be described with reference to embodiments or examples and the figures of the accompanying drawings pages.

### BRIEF DESCRIPTION OF DRAWINGS

FIG. 1: Schematic overview of the in-vitro experimental design, (a) refers to the live infectious OsHV-1, (b) whole virus killed by chemical (BEI, Formaldehyde), heat inactivated, or exposed to freeze-thaw cycles; (c) OsHV-1 DNA, (d) OsHV-1 whole protein extract, (e) Poly (I:C, positive control), (f) sterile filtered seawater (negative control). ROS refers to “reactive oxygen species”, ORF to “open reading frame”.

FIG. 2: Viability (in percent) of Crassostrea gigas hemocytes exposed for 1 h to OsHV-1 which had been inactivated with different preparations of A) Binary ethylenimine BEI, B) formaldehyde, and C) temperature treatments. Panel D) shows viability of hemocytes exposed to two viral protein concentrations and purified viral DNA (See Table 1 for details of the different treatments). Box plots indicate the median, upper and lower quartiles; whiskers indicate the highest and the lowest percent viable hemocytes and dots indicate outliers. * (p<0.05), ** (p<0.01), *** (p<0.001), **** (p<0.0001). These box plots represent data from both experiment 1 and 2 (n=6), except the antigen preparations indicated by a diamond shape on the boxplots, which have been tested in experiment 1 only (n=3).

FIG. 3: Reactive oxygen species (ROS) production in Crassostrea gigas hemocytes exposed for 1 h to OsHV-1 which had been inactivated with different preparations of A) Binary ethylenimine BEI, B) formaldehyde, and C) temperature treatments. Panel D) shows ROS production in hemocytes exposed to two viral protein concentrations and purified viral DNA (See Table 1 for details of the different treatments). Box plots indicate the median, upper and lower quartiles; whiskers indicate the highest and the lowest values of the dataset and dots indicates outliers. * (p<0.05), ** (p<0.01), *** (p<0.001), (p<0.0001). These box plots represent data from both experiment 1 and 2 (n=6), except the antigen preparations indicated by a diamond shape on the boxplots, which have been tested in experiment 1 only (n=3).

FIG. 4: Heatmap focusing on the expression of 5 genes related innate immune response in Crassostrea gigas and ORF87, an OsHV-1 ORF expressed early after infection. The intensities of the colours indicate the magnitude of the differential expression (log 2 fold-change). The fold-changes were calculated by comparing the expression of each gene in hemocytes exposed to Poly (I:C), live infectious OsHV-1, or OsHV-1 inactivated, with its expression in negative control (hemocytes exposed to SSW). Significance levels are expressed by asterisks: * (p<0.05), ** (p<0.01), *** (p<0.001), **** (p<0.0001).

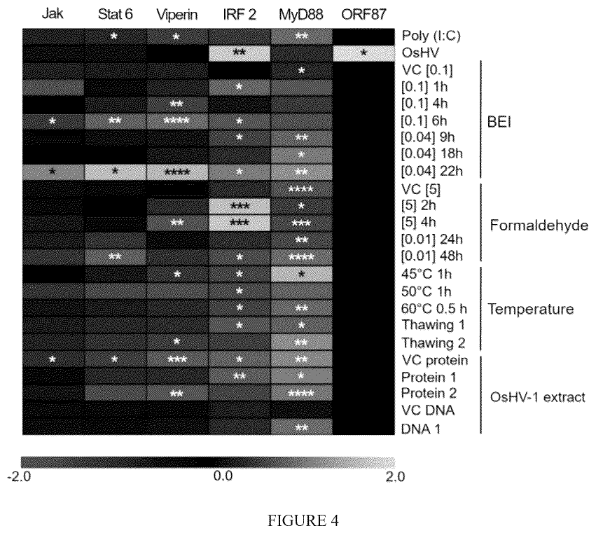

### DETAILED DESCRIPTION

Infectious diseases are a major constraint to the expansion of shellfish production worldwide. Pacific oyster mortality syndrome (POMS), a polymicrobial disease triggered by the Ostreid herpesvirus-1 (OsHV-1), has devastated the global Pacific oyster (Crassostrea gigas) aquaculture industry. Recent ground-breaking research revealed that C. gigas possess an immune memory, capable of adaption, to improve immune response upon a second exposure to a pathogen. This paradigm shift opens the door for developing ‘vaccines’ to improve shellfish survival during disease outbreaks.

In the present study, we developed an in-vitro assay using hemocytes—the main effectors of the C. gigas immune system—collected from juvenile oysters susceptible to OsHV-1. The potency of multiple antigen preparations (e.g., chemically, and physically inactivated OsHV-1, viral DNA, and protein extracts) to stimulate an immune response in hemocytes was evaluated using flow-cytometry and droplet digital PCR to measure immune-related subcellular functions and gene expression, respectively. Immune response to the different antigens was benchmarked against that of hemocytes treated with Poly (I:C). We identified 10 antigen preparations capable of inducing immune stimulation in hemocytes (ROS production and positively expressed immune related genes) after 1 h of exposure, without causing cytotoxicity. These findings are significant as they evidence the potential for priming oyster innate immunity using viral antigens which might enable cost-effective therapeutic treatment to mitigate OsHV-1/POMS. Further testing of these antigen preparations using an in-vivo infection model is essential to validate promising candidate vaccines.

### Materials and Methods

In June 2021, two separate experiments were conducted to (1) screen multiple preparations of inactivated OsHV-1 (hereafter referred to as antigens) by measuring immune-related functions of hemocytes using flow cytometry (FCM), and (2) confirm the potency of a selected subset of antigen preparations to stimulate immunity using FCM (cytotoxicity and ROS) and molecular analyses (immune related gene expression).

### 1 Preparation of OsHV-1 Antigens

### 1.1 OsHV-1 Stock

The OsHV-1 suspension stock was produced in October 2019 as described in Camara et al. (2017). Briefly, tissue from high virus load oysters was homogenized, purified by serial filtrations down to 0.22 μm and cryopreserved according to Kirkland et al. (2015). On the 3 Jun. 2021, cryopreserved OsHV-1 stock was defrosted by dipping in 22° C. water bath for 10 min. Viral suspension was titrated using qPCR (Martenot et al., 2010) and diluted in 0.22 μm-filtered sterile seawater (SSW) to reach a final concentration of 9.0. 105 copies. μl-1. Prepared virus was then inactivated using the following methods.

### 1.2 BEI Inactivation

A 0.2M Binary ethylenimine (BEI; CAS RN 151-56-4) solution was prepared by cyclisation of 0.2M 2-bromoethylamin-HBr in 0.2M NaOH at 37° C. for 1 h (Bahnemann, 1990). The BEI solution was added to OsHV-1 suspension to either a concentration of 0.1% (v/v) and incubated at 22° C. for 1 h, 4 h, 6h, or to a concentration of 0.04% (v/v) and incubated at 22° C. for 4 h, 6 h, 9 h, 18 h, 22h. Inactivation reactions were stopped by addition of sodium thiosulphate 1M (neutralizing agent) to reach a final concentration of 10% (v/v). Obtained inactivated viral suspensions were stored at 4° C. until use. Suspension of neutralised 0.1% (v/v) BEI diluted in filtered (0.22 μm) SSW was used as the vehicle control (Table 1).

### 1.3 Formaldehyde Inactivation

Formaldehyde solution 37% (w/v) was added to the OsHV-1 suspension to a final concentration of 5%, 0.3% or 0.01%, and incubated at 22° C. for 2 h, 4 h, 12 h (5%); for 4 h, 8h, 12 h, 24 h, 48 h, 60h (0.3%); or for 12 h, 24 h, 60h (0.01%), respectively. Virus inactivation was stopped by addition of 35% sodium bisulphite to reach a final concentration of 0.035%, and suspensions were then stored at 4° C. Suspension of 5% (v/v) neutralised formalin diluted in filtered (0.22 μm) SSW was used as the vehicle control (Table 1).

### 1.4 Heat Inactivation

For preparation of heat-inactivated OsHV-1, viral suspensions were incubated for 1 h at 45° C., 50° C., 52° C. or for 30 min at 54° C., 56° C. and 60° C. using a dry bath, and then stored at 4° C. until use (Table 1).

### 1.5 Freeze-Thaw Cycles

Viral suspensions were placed at −80° C. for 12 h, transferred to −20° C. for 12 h, and then maintained at 4° C. After complete thawing, viral suspensions were immediately re-frozen at −80° C. for 12 h. The freeze-thaw cycles were repeated twice (Thawing 1) or 3 time for (Thawing 2), and the antigen suspensions were stored at 4° C. until use (Table 1).

### 1.6 OsHV-1 DNA

Total viral DNA was extracted from 1 ml of the OsHV-1 stock suspension using blood and tissues kit (QIAGEN) according to the manufacturer's protocol. Extracted DNA was resuspended in 10 mM Tris-HCl buffer (pH 7.4) to reach a final concentration of 10 ng DNA. μl-1 and stored at −20° C. until use. The solution 10 mM Tris-HCl buffer (pH 7.4) was used as a vehicle control (Table 1).

### 1.7 Viral Proteins

Total viral proteins were extracted from 5 ml of the OsHV-1 stock suspension via bead beating for 10 min at 1500 rpm and 4° C., using a 1600 MiniG automated tissue homogenizer (SPEX Sample Prep, Metuchen, NJ). Proteins were then solubilised for 45 min by adding 2 ml of extraction buffer (100 mM potassium phosphate, 50 mM NaCl, 0.1 mM EDTA-Na2, 1% polyvinyl pyrrolidone, 2 mM phenylmethylsulfonyl fluoride and 0.1% TritonX-100; pH 7.5 at 4° C.). Solubilized proteins were extracted by centrifugation at 13,500 rpm for 15 min at 4° C. and ultra-filtered (10 kDa molecular weight cut-off, Amicon Ultra-0.5 10K, Merk-Millipore, Burlington, USA) following manufacturer's specifications. The semi-purified proteins were reconstituted in 200 μl of sterile Phosphate Buffer Saline (PBS). Total protein content of the lysate was quantified by the Lowry protein assay (Fryer et al., 1986), diluted in sterile PBS to reach a final concentration of 0.2 mg·ml-1 and stored at −80° C. until use. Protein extracts were either pure (Protein 1) or diluted at 1:10 (v/v) in PBS (Protein 2). Sterile PBS was used as a vehicle control (Table 1).

### 2 Oysters

Experiments were performed using hemolymph of hatchery-bred juvenile Crassostrea gigas [8 months old, mean live weight 6.7±3.1 g]. These oysters were the offspring of naïve wild stocks and therefore expected to be highly susceptible to POMS. Prior to sampling, oysters were maintained in flow-through seawater (10 μm filtered) at ambient conditions (10-22° C. and a salinity of 35±1) and fed ad libitum with hatchery-grown algal food. Experimental oysters were considered naïve to POMS/OsHV-1 due to their rearing with continuous supply of UV-sterilized seawater (80 mJ cm-2) and maintenance under strict biosecurity management to ensure they remained OsHV-1-free. This status was confirmed prior to the experiment by the absence of significant mortality and OsHV-1 DNA detection in tissue (n=10). Oysters were starved for 24 h prior to hemolymph collection, to minimise algal contamination of the hemolymph.

TABLE 1
Summary of the different methods of inactivation and antigen preparation
including inactivation agents and corresponding final concentrations, exposure
time, neutralisation agents and corresponding final concentrations.

| | [inactivation | Exposure | Neutralisation | [Neutralisation |
|---|---|---|---|---|
| Inactivation method | agent] | time | agent | agent] |
| Binary ethylenimine | 0.10% | 1 h | Sodium | 10% |
| (BEI) 0.2M | | 4 h | thiosulphate | |
| | | 6 h | 1M | |
| | 0.04% | 4 h | | |
| | | 6 h | | |
| | | 9 h | | |
| | | 18 h | | |
| | | 22 h | | |
| Formaldehyde solution | 5% | 2 h | Sodium | 0.035% |
| (37% (w/v)) | | 4 h | bisulphite | |
| | | 12 | | |
| | 0.30% | 4 h | | |
| | | 8 h | | |
| | | 12 h | | |
| | | 24 h | | |
| | | 48 h | | |
| | | 60 h | | |
| | 0.01% | 12 h | | |
| | | 24 h | | |
| | | 48 h | | |
| | | 60 h | | |
| Heat inactivation | Heat shock 45° C. | | 1 h | |
| | Heat shock 50° C. | | | |
| | Heat shock 52° C. | | | |
| | Heat shock 54° C. | | 0.5 h | |
| | Heat shock 56° C. | | | |
| | Heat shock 60° C. | | | |
| Freeze-thaw cycles | (−80/−20/4/−80) × 2 | | | |
| | (−80/−20/4/−80) × 3 | | | |
| Virus extract | Protein extract 1 | | | |
| | Protein extract 2 | | | |
| | DNA extract 1 | | | |
| | DNA extract 2 | | | |

### 3 Experiment 1: Screening of Antigen Preparations Using Flow Cytometry (FCM).

### 3.1 Hemolymph Collection

Between the 14th and 16th June 2021, hemolymph samples of between 22 and 35 oysters were collected and pooled daily for experimental exposure. A small notch was made in the shell using wire cutters and oysters were bled from the adductor muscle sinus using a 25G 1.5-inch needle with 1 mL syringe, previously kept on ice. Between 150 and 1500 μl of hemolymph was withdrawn from each individual and immediately added to a 1.5 mL Eppendorf, previously kept on ice. Individual samples were checked under a light microscope (40× magnification) to confirm purity. When ~9 mL of pure, clean hemolymph had been collected from multiple individuals, hemolymph samples were pooled, diluted (1:4 v/v) with autoclaved 0.2 μm-filtered sterile seawater (FSSW) and stored on ice until exposure.

### 3.2 In-Vitro Exposure

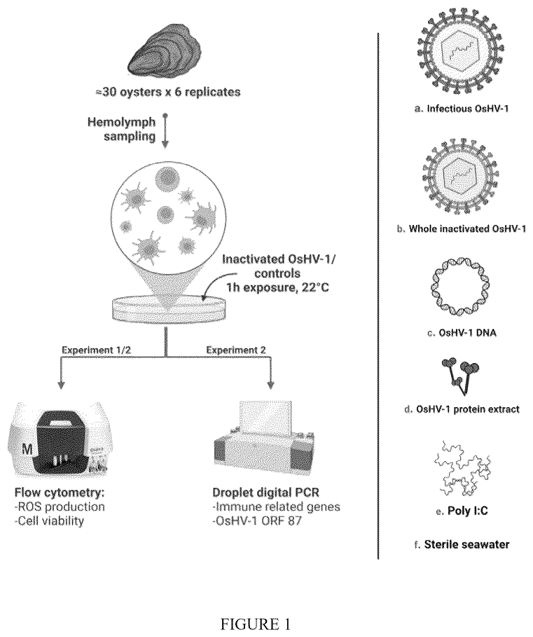

Before experimental exposure, each antigen preparation and vehicle control were diluted (1:100 v/v) with FSSW. Polyinosinic-polycytidylic acid [Poly (I:C)], a synthetic analogue of double-stranded RNA (dsRNA), was used as positive control (0.05 mg·ml-1 in SSW, Green et al., 2015b) and FSSW was used as a negative control. Antigen preparations or controls were added (1:80 v/v) in hemolymph (detailed above) and incubated at room temperature (22° C.) for 1h. Following experimental exposure, hemocyte reactive oxygen species (ROS) production and viability were determined (detailed below). This process was repeated daily for 3 consecutive days to obtain n=3 independent replicates (FIG. 1, Experiment 1).

### 3.3 FCM Assessments

Hemolymph samples which had been exposed to different antigen preparations for 1 h were analysed using a Guava® EasyCyte™ 5HT flow cytometer equipped with a blue laser (488 nm) and green (525/30 nm), yellow (583/26 nm) and red (695/50) detectors (EMD Millipore, USA). Samples were mixed at medium speed and acquired at a flow rate of 0.24 μL second-1 for 30 seconds.

The production of intracellular reactive oxygen species (ROS) was measured using 2′,7′-Dichlorodihydro-fluorescein diacetate (DCFH-DA, Sigma Aldrich, D6883) according to Donaghy et al, (2012). Following 30 minutes of experimental treatments incubation hemocyte samples were stained with 10 μM final concentration of DCFH-DA and incubated in the dark at room temperature (22° C.) for another 30 minutes until analysis (=total 1 h exposure to treatments). Relative ROS production was expressed as the level of green (FL1) fluorescence.

The viability of hemocytes was measured using Fluorescein diacetate (FDA, Invitrogen, F1303) according to Rolton et al. (2020). Following 50 minutes of experimental treatments incubation, hemocytes were stained with 1.25 mg L-1 final concentration of FDA and incubated in the dark at room temperature for 10 minutes until analysis (=total 1 h exposure to treatments). Hemocytes were divided into those with high FL1 (corresponding to metabolically active/viable cells) and those with low green fluorescence (non-viable).

### 4 Experiment 2: Validation of Antigen Preparations Using FCM and Molecular Analyses.

Antigen preparations that induced ROS production in hemocytes without cytotoxicity during experiment 1 were further tested in experiment 2, and their potency validated using FCM and molecular analyses. Specific antigen preparations which induced significantly less ROS production during experiment 1 were also tested again to maintain a range of contrasted immune responses and improve validation.

Between 17th and 19th June 2021, hemolymph samples of 22-28 oysters were collected daily (as detailed above) until 16 ml of pure, clean hemolymph had been collected, and pooled. As previously described, each antigen preparation and vehicle control was diluted (1:100 v/v) with FSSW. For flow cytometry, antigen preparations or controls were added (1:80 v/v) to (1:4 v/v) diluted hemolymph (detailed above) and incubated at room temperature (22° C.) for 1h. Reactive oxygen species (ROS) production and viability of hemocytes were determined as described in Experiment 1. Concomitantly, the expression of 5 immune-related genes and the viral gene ORF 87 were evaluated by adding 20 μl of the diluted antigen preparations (1:100 (v/v), see above) or controls to 400 μl of pure hemolymph in 1.5 ml Eppendorf tubes and incubated at 22° C. for 1h. This process was repeated daily for 3 consecutive days to obtain n=3 independent replicates (FIG. 1, experiment 2).

### 4.1 Molecular Analyses

RNA was extracted from 400 μl of hemolymph previously exposed to 20 μl of antigen preparation, SSW (negative control), Poly I:C (positive control) or live infectious OsHV-1 using the Quick RNA/DNA Miniprep plus kit (Zymo Research) according to the manufacturer protocol. RNA was eluted in 50 μl DNAse/RNase-free water. As described in Delisle et al, (2022), samples were treated with DNAse I (TURBO™ DNase, Invitrogen), the absence of DNA in the samples was confirmed by a 16S PCR assay, purity of the isolated RNA was assessed, and DNAse-treated RNA was transcribed into cDNA. Finally, droplet digital PCR (ddPCR) was conducted in an automated droplet generator (QX200 Droplet Digital PCR System™, Bio-Rad) to determine the expression of five genes (Jak, Stat6, Viperin, IRF2, Myd88) related to oyster innate immunity (Green and Montagnani, 2013; He et al., 2015; Rosani et al., 2015), as well as the ORF 87, an OsHV-1 gene selected from the 39 ORFs described by Segarra et al. (2014). Each ddPCR reaction included 1 μl of 3 μM of the primers (Jak, Stat, Viperin, Myd88) or 10 μM (IRF2, ORF87), 10 μl ddPCR Supermix for Evagreen (Bio-Rad), 2 μl cDNA and 7 μl sterile water for a total reaction volume of 21 μl. As described in Delisle et al., (2022) ddPCR was performed using the following cycling protocol: hold at 95° C. for 5 s, 45 cycles of 95° C. for 30 s, 60° C. 1 min, 4° C. for 5 min and a final enzyme deactivation step at 90° C. for 5 min. The plate was then analysed on the QX200 instrument (Bio-Rad). For each ddPCR plate run, at least one negative control (RNA/DNA-free water; Life Technologies), and one positive control (C. gigas DNA or Gblock for ORF87 diluted 1/10,000) were included.

### 5 Statistical Analyses

Statistical analyses were computed using R 4.2.1 (https://www.r-project.org/) and the packages “ggpubr” (Kassambara, 2020) and “rstatix” (Kassambara, 2021). One way ANOVA and t tests were performed to evaluate the effects of each antigen preparation on ROS production, hemocytes viability and gene expression, compared with the effects of SSW exposure (negative control), and p-values were adjusted with Holm correction. For gene expression, a heatmap was constructed using Multiple Experiment Viewer software (Saeed et al., 2003) http://mev.tm4.org/#/datasets/upload). For all analyses, the threshold significance level was set at 0.05.

### 6 Results

### 6.1 79% of the Antigenic Preparations Did not Affect Hemocytes Viability.

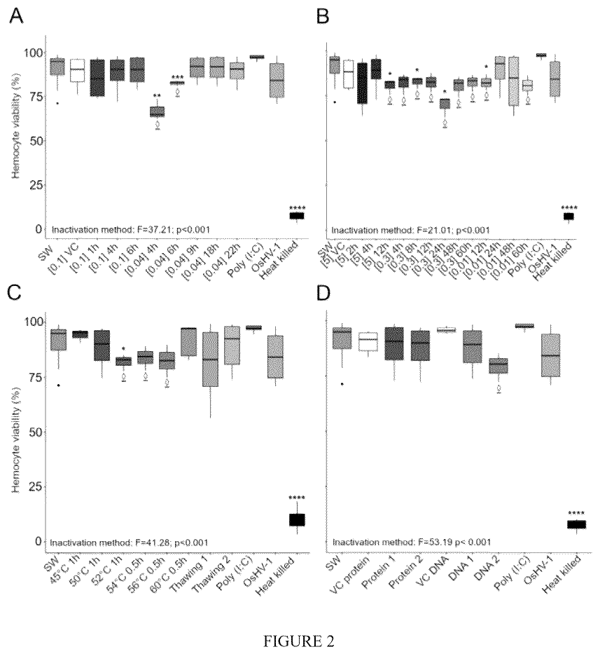

Of the 33 antigenic preparations tested during experiments 1 and 2, 26 had no cytotoxic effect on oyster hemocytes (as measured by viability). However, hemocytes exposed for 1 h to the following antigen preparations—OsHV-1 previously inactivated using BEI 0.04% for 4 h and 6h (FIG. 2A), formaldehyde 5% for 12 h, formaldehyde 0.3% for 8 h, 24 h, formaldehyde 0.01% for 12 h, (FIG. 2C) and heated at 52° C. for 1h (FIG. 2D)—showed a significant reduction in viability compared to hemocytes exposed to sterile seawater (SSW). The viability of hemocytes that had been heat killed (negative control) was very low (10.2±5.2% mean±SD, n=3) compared to hemocytes exposed to SSW (91.3±8.0%, p=1.7e-13) (FIG. 2).

### 6.2 Antigenic Preparations Induced ROS Production in Hemocytes

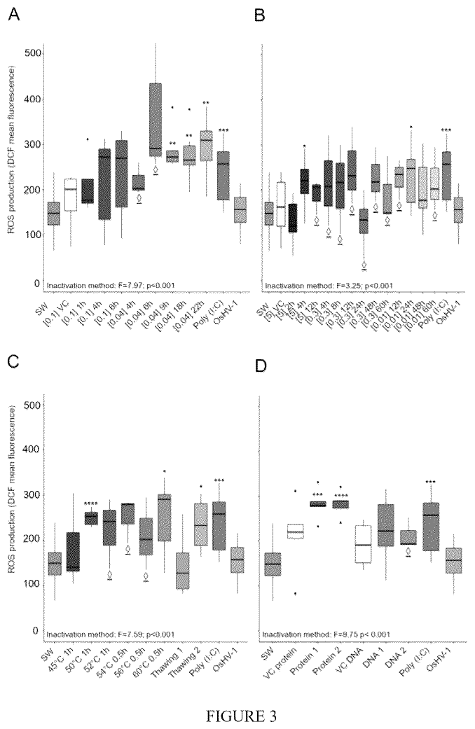

Hemocytes of C. gigas which had been exposed to ten antigenic preparations of inactivated OsHV-1 and to the positive control (Poly I:C) showed increased ROS production (FIG. 3). Specifically, preparations of virus inactivated using BEI 0.04% at 9, 18 and 22h (FIG. 3A), formaldehyde 5% 4h, formaldehyde 0.01% 24 h (FIG. 3B), heat shock at 50° C. for 1 h, at 60° C. for 0.5h, or 3 freeze-thaw cycles (FIG. 3C) as well as viral protein extracts pure or diluted 1/10 (v/v, FIG. 3D) all significantly increased hemocyte ROS production compared to those exposed to SSW (p<0.001).

Based on an absence of cytotoxicity and high levels of ROS production (using FCM), ten antigen preparations, as well as eight additional antigen preparations which induced a limited subcellular immune response (FIGS. 2 and 3), were selected for validation (using FCM and molecular analysis).

### 6.3 all the Selected Antigen Preparations Induced Up-Regulation of Immune Related Genes.

All the selected antigen preparations induced the upregulation of at least one of the immune related genes of C. gigas. Exposure of hemocytes to the Poly (I:C) at 0.05 mg. ml-1 for 1 h induces a significant upregulation of MyD88, Viperin and Stat6. Exposure of hemocytes to OsHV-1 which had been inactivated using BEI 0.04% for 22 h resulted in a significant upregulation of all 5 of the immune related genes analysed. Vehicle controls did not induce the expression of the immune related genes in hemocytes, except the phosphate buffered saline (VC protein, Table 1) which induced a significant upregulation of Stat6, Viperin, IRF2 and MyD88. As expected, the expression of ORF87 was only detected in hemocytes exposed to live infectious OsHV-1.

### 7 Discussion

In the present study, we were able to stimulate an antiviral response in juvenile Crassostrea gigas hemocytes using inactivated OsHV-1 and viral extracts, as shown by ROS induction and upregulation of antiviral response-related genes. A previous study, in which C. gigas hemocytes were exposed to live OsHV-1, also resulted in the expression of genes involved in immune-related functions (Morga et al., 2017); however, this is the first-time inactivated OsHV-1 preparations have been shown to elicit an immune response in-vitro.

Virus inactivation transforms antigens from being infectious to non-infectious and it is important to determine any cytotoxicity of the antigen preparations prior to determining their potency (Bahnemann, 1990). In the absence of bivalve cell line cultures in which to determine cytotoxicity, here, we used a FCM-based assessment of hemocyte viability for rapid in-vitro screening of vaccine preparations. Morga et al (2009), similarly used FCM to determine hemocyte viability following an in-vitro exposure of C. gigas hemocytes to the protozoan parasite, Bonamia ostreae. Among the 33 antigen preparations tested in our study, only 7 induced a significant decline in hemocyte viability. Incomplete neutralisation of the denaturing agent could explain this observed cytotoxicity. The residual infectivity post-inactivation of the preparations tested was verified by the absence of the open reading frame 87 (ORF87) expression in the hemocytes following one hour of antigen exposure. This ORF codes for an apoptosis inhibitor protein and is expressed during the first hours post OsHV-1 infection in oysters (Segarra et al., 2014; Morga et al., 2017).

A one-hour exposure of hemocytes to 0.5 mg·ml-1 of Poly (I:C) was sufficient to induce cellular ROS production—a proxy of immune response in bivalves (de la Ballina et al., 2022)—and an upregulation of the genes coding key antiviral effectors: MyD88, Stat 6 and Viperin, validating our screening approach. An induction of MyD88, an essential signal transducer in the interleukin-1 and Toll-like receptor signalling pathways, was also observed in mussel (Mytilus galloprovincialis) and scallop (Pecten maximus) hemocytes which had been stimulated for 8 h and 3 h respectively with 50 μg·ml-1 of Poly (I:C), a much higher concentration than was used in the present study (Pauletto et al., 2014; Moreira et al., 2020). Upregulation of Viperin—an interferon-inducible antiviral protein—and Stat 6—a signal transducer and activator of transcription—have also been observed in oyster hemocytes primed with Poly (I:C) compared to those exposed to seawater (Green et al., 2015c, 2016).

The interaction between OsHV-1 and the oyster host cells has not been fully elucidated and the functions of proteins coded by OsHV-1 genome are largely unknown. However, antibody blocking, and pull-down assays suggest the potential implication of three putative OsHV-1 membrane proteins (ORF 25, ORF 41 and ORF 72) in the virus/host interaction by binding of host cytoskeleton (Martenot et al., 2019; Yu et al., 2021). Interestingly, antigen preparations resulting from BEI inactivation induced the strongest immune and antiviral response. Binary ethylenimine is an aziridine preparation commonly used in veterinary vaccine production as an inactivating agent (Zhu et al., 2018). At 1 mM, BEI induces an alkylation of the nucleic acids without damaging proteins (Groseil et al., 1995). In the present work, high ROS production coupled with upregulation of the five tested genes were recorded when using OsHV-1 inactivated with BEI at [0.04%] for 22h, supporting the role of viral proteins in the stimulation of antiviral response in oysters. Conversely, we observed a reduced potency of antigen preparations that were inactivated by formaldehyde, one of the most widely used inactivating agents, for an extended exposure time (12 and 48h) suggesting an irreversible denaturation of proteins induced by formaldehyde (Sanders et al., 2015).

Three antigen preparations inactivated with BEI induced a strong upregulation of the transcript coding for Viperin. Viperin is a highly conserved evolutionary host protein (Fitzgerald, 2011), which restricts the replication of a range of RNA and DNA viruses [e.g., human cytomegalovirus (Chin and Cresswell, 2001), immunodeficiency virus (Nasr et al., 2012), and Hepatitis C virus (Helbig et al., 2005)], by interacting with viral protein and altering the site of virus budding. In C. gigas, Viperin has been reported to be one of the earliest and most regulated genes in response to OsHV-1 exposure (Green et al., 2013, 2015b) and also exhibits the same level of antiviral activity as human Viperin against Dengue virus in-vitro (Green et al., 2013, 2015c). Consequently, its expression in hemocytes could constitute a good indicator of the potency of the antigen preparations.

Besides chemical agents, physical methods were used to inactivate OsHV-1 in this study. Thermal inactivation of OsHV-1 at 50° C. and 60° C. and long thawing cycles caused an increase in ROS production while the associated molecular responses appeared moderate. These variations in the results could probably be explained by the broad range of stressors able to induce ROS production in bivalves (Donaghy et al., 2015) compared to the specificity of the antiviral response. Nonetheless, we obtained a good correlation overall between immune-related markers measured via flow-cytometry (ROS production) and qPCR analyses (immune-gene expression), with the ranking of the ten best antigen preparations maintained when up-regulation of immunity genes was considered.

Of note, protein extracts obtained from OsHV-1 and resuspended in PBS also induced a marked positive expression of MyD88, IRF2 and Viperin; however, the confounding effect of the vehicle control PBS on the hemocytes cannot be excluded. Similar induction of immune response following PBS exposure has been reported (Catalán et al., 2012; Gálvez et al., 2020).

To conclude, these findings are significant as they evidence for the first time the potential for stimulating oyster's innate immunity using viral antigens which might enable cost-effective therapeutic treatment to mitigate the economic impacts of OsHV-1/POMS. For instance, chemical inactivation using the Binary ethylenimine at 0.04% for 22 h was identified as the best candidate preparation.

Although the invention has been described with reference to embodiments or examples it should be appreciated that variations and modifications may be made to these embodiments or examples without departing from the scope of the invention. Where known equivalents exist to specified elements, features, integers, or other limitations, of the matter described in the Summary of Invention, such equivalents are incorporated as if specifically referred to in this specification. Variations and modifications to the embodiments or examples that include elements, features, integers, or other limitations, disclosed in and selected from the referenced publications are within the scope of the invention unless specifically disclaimed. The advantages provided by the invention and discussed in the description included in this specification may be provided in the alternative or in combination in these different embodiments of the invention.

### REFERENCED PUBLICATIONS

Agius, J. R., Corbeil, S., and Helbig, K. J. (2020). Immune control of herpesvirus infection in molluscs. Pathogens 9, 1-11. doi: 10.3390/pathogens9080618.
Allam, B., and Ford, S. E. (2006). Effects of the pathogenic Vibrio tapetis on defence factors of susceptible and non-susceptible bivalve species: Haemocyte changes following in vitro challenge. Fish Shellfish Immunol. 20, 374-383. doi: https://doi.org/10.1016/j.fsi.2005.05.012.
Allam, B., and Raftos, D. (2015). Immune responses to infectious diseases in bivalves. J. Invertebr. Pathol. 131, 121-136. doi: 10.1016/j.jip.2015.05.005.
Bahnemann, H. G. (1990). Inactivation of viral antigens for vaccine preparation with particular reference to the application of binary ethylenimine. Vaccine 8, 299-303. doi: 10.1016/0264-410X (90) 90083-X.
Barbosa-Solomieu, V., Dégremont, L., Vázquez-Juárez, R., Ascencio-Valle, F., Boudry, P., and Renault, T. (2005). Ostreid Herpesvirus 1 (OsHV-1) detection among three successive generations of Pacific oysters (Crassostrea gigas). Virus Res. 107, 47-56. doi: 10.1016/j.viruses.2004.06.012.
Bøgwald, J., and Dalmo, R. A. (2019). Review on Immersion Vaccines for Fish: An Update 2019. Microorganisms 7. doi: 10.3390/microorganisms7120627.
Buchmann, J. P., and Holmes, E. C. (2015). Cell Walls and the Convergent Evolution of the Viral Envelope. Microbiol. Mol. Biol. Rev. 79, 403-418. doi: 10.1128/MMBR.00017-15.Address.
Buchmann, K. (2014). Evolution of innate immunity: clues from invertebrates via fish to mammals. Front. Immunol. 5, 1-8. doi: 10.3389/fimmu.2014.00459.
Burge, C. A., Friedman, C. S., Kachmar, M. L., Humphrey, K. L., Moore, J. D., and Elston, R. A. (2021). The first detection of a novel OsHV-1 microvariant in San Diego, California, USA. J. Invertebr. Pathol. 184, 107636. doi: https://doi.org/10.1016/j.jip.2021.107636.
Burge, C. A., Mark Eakin, C., Friedman, C. S., Froelich, B., Hershberger, P. K., Hofmann, E. E., et al. (2014). Climate Change Influences on Marine Infectious Diseases: Implications for Management and Society. Ann. Rev. Mar. Sci. 6, 249-277. doi: 10.1146/annurev-marine-010213-135029.
Camara, M. D., Yen, S., Kaspar, H. F., Kesarcodi-Watson, A., King, N., Jeffs, A. G., et al. (2017). Assessment of heat shock and laboratory virus challenges to selectively breed for ostreid herpesvirus 1 (OsHV-1) resistance in the Pacific oyster, Crassostrea gigas. Aquaculture 469, 50-58. doi: 10.1016/j.aquaculture.2016.11.031.
Catalán, T. P., Wozniak, A., Niemeyer, H. M., Kalergis, A. M., and Bozinovic, F. (2012). Interplay between thermal and immune ecology: Effect of environmental temperature on insect immune response and energetic costs after an immune challenge. J. Insect Physiol. 58, 310-317. doi: 10.1016/j.jinsphys.2011.10.001.
Cheng, T. (1996). “Hemocytes: forms and functions.,” in The Eastern Oyster Crassostrea virginica. (College Park, MD, USA), 299-333.
Chin, K. C., and Cresswell, P. (2001). Viperin (cig5), an IFN-inducible antiviral protein directly induced by human cytomegalovirus. Proc. Natl. Acad. Sci. U.S.A. 98, 15125-15130. doi: 10.1073/pnas.011593298.
Cong, M., Song, L., Wang, L., Zhao, J., Qiu, L., Li, L., et al. (2008). The enhanced immune protection of Zhikong scallop Chlamys farreri on the secondary encounter with Listonella anguillarum. Comp. Biochem. Physiol. Part B Biochem. Mol. Biol. 151, 191-196. doi: https://doi.org/10.1016/j.cbpb.2008.06.014.
Dadar, M., Dhama, K., Vakharia, V. N., Hossein, S., Karthik, K., Tiwari, R., et al. (2016). Advances in Aquaculture Vaccines Against Fish Pathogens: Global Status. Rev. Fish. Sci. Aquac. 0, 1-34. doi: 10.1080/23308249.2016.1261277.
Daszak, P., Cunningham, A. A., and Hyatt, A. D. (2000). Emerging infectious diseases of wildlife-Threats to biodiversity and human health. Science (80-.). 287, 443-449. doi: 10.1126/science.287.5452.443.
De la Ballina, N. R., Maresca, F., Cao, A., and Villalba, A. (2022). Bivalve Haemocyte Subpopulations: A Review. Front. Immunol. 13. doi: 10.3389/fimmu.2022.826255.
De Lorgeril, J., Lucasson, A., Petton, B., Toulza, E., Montagnani, C., Clerissi, C., et al. (2018). Immune-suppression by OsHV-1 viral infection causes fatal bacteraemia in Pacific oysters. Nat. Commun. 9, 4215. doi: 10.1038/s41467-018-06659-3.
Dégremont, L., Nourry, M., and Maurouard, E. (2015). Mass selection for survival and resistance to OsHV-1 infection in Crassostrea gigas spat in field conditions: response to selection after four generations. Aquaculture 446, 111-121. doi: https://doi.org/10.1016/j.aquaculture.2015.04.029.
Delisle, L., Laroche, O., Hilton, Z., Burguin, J., Rolton, A., Berry, J., et al. (2022). Understanding the dynamic of POMS infection and the role of microbiota composition in the survival of Pacific oysters, Crassostrea gigas. Microbiol. Spectr. doi: https://doi.org/10.21203/rs.3.rs-1636731/v1.
Divilov, K., Schoolfield, B., Morga, B., Degremont, L., Burge, C. A., Mancilla Cortez, D., et al. (2019). First evaluation of resistance to both a California OsHV-1 variant and a French OsHV-1 microvariant in Pacific oysters. BMC Genet. 20, 96. doi: 10.1186/s12863-019-0791-3.
Donaghy, L., Hong, H. K., Jauzein, C., and Choi, K. S. (2015). The known and unknown sources of reactive oxygen and nitrogen species in haemocytes of marine bivalve molluscs. Fish Shellfish Immunol. 42, 91-97. doi: 10.1016/j.fsi.2014.10.030.
Donaghy, L., Kraffe, E., Le Goïc, N., Lambert, C., Volety, A. K., and Soudant, P. (2012). Reactive Oxygen Species in Unstimulated Hemocytes of the Pacific Oyster Crassostrea gigas: A Mitochondrial Involvement. PLOS One 7, 1-10. doi: 10.1371/journal.pone.0046594.
EFSA (2010). Scientific opinion on the increased mortality events in Pacific oysters. Eur. Food Saf. Auth. J. 8, 1-60. doi: 10.2903/j.efsa.2010.1894.
EFSA (2015). Oyster mortality. EFSA J. 13, 4122. doi: 10.2903/j.efsa.2015.4122.
Fitzgerald, K. A. (2011). The interferon inducible gene: Viperin. J. Interf. Cytokine Res. 31, 131-135. doi: 10.1089/jir.2010.0127.
Gálvez, D., Añino, Y., Vega, C., and Bonilla, E. (2020). Immune priming against bacteria in spiders and scorpions? PeerJ 2020, 0-14. doi: 10.7717/peerj.9285.
Green, T. J., Helbig, K., Speck, P., and Raftos, D. A. (2016). Primed for success: Oyster parents treated with poly(I:C) produce offspring with enhanced protection against Ostreid herpesvirus type I infection. Mol. Immunol. 78, 113-120. doi: 10.1016/j.molimm.2016.09.002.
Green, T. J., and Montagnani, C. (2013). Poly I:C induces a protective antiviral immune response in the Pacific oyster (Crassostrea gigas) against subsequent challenge with Ostreid herpesvirus (OsHV-1 μvar). Fish Shellfish Immunol. 35, 382-388. doi: 10.1016/j.fsi.2013.04.051.
Green, T. J., Montagnani, C., Benkendorff, K., Robinson, N., and Speck, P. (2013). Ontogeny and water temperature influences the antiviral response of the Pacific oyster, Crassostrea gigas. Fish Shellfish Immunol. 36, 151-157. doi: 10.1016/j.fsi.2013.10.026.
Green, T. J., Raftos, D., Speck, P., and Montagnani, C. (2015a). Antiviral immunity in marine molluscs. J. Gen. Virol. 96, 2471-2482. doi: 10.1099/jgv.0.000244.
Green, T. J., Rolland, J. L., Vergnes, A., Raftos, D., and Montagnani, C. (2015b). OsHV-1 countermeasures to the Pacific oyster's anti-viral response. Fish Shellfish Immunol. 47, 435-443. doi: 10.1016/j.fsi.2015.09.025.
Green, T. J., Speck, P., Geng, L., Raftos, D., Beard, M. R., and Helbig, K. J. (2015c). Oyster viperin retains direct antiviral activity and its transcription occurs via a signaling pathway involving a heat-stable haemolymph protein. J. Gen. Virol. 96, 3587-3597. doi: 10.1099/jgv.0.000300.
Groseil, C., Guerin, P., and Adamowicz, P. (1995). Evaluation by polymerase chain reaction on the effect of betapropiolactone and binary ethyleneimine on DNA. Biologicals 23, 213-220. doi: 10.1006/biol.1995.0035.
Gutierrez, A. P., Symonds, J., King, N., Steiner, K., Bean, T. P., and Houston, R. D. (2020). Potential of genomic selection for improvement of resistance to ostreid herpesvirus in Pacific oyster (Crassostrea gigas). Anim. Genet. 51, 249-257. doi: https://doi.org/10.1111/age.12909.
Harvell, C. D., Montecino-Latorre, D., Caldwell, J. M., Burt, J. M., Bosley, K., Keller, A., et al. (2019). Disease epidemic and a marine heat wave are associated with the continental-scale collapse of a pivotal predator (Pycnopodia helianthoides). Sci. Adv. 5, 1-9. doi: 10.1126/sciadv.aau7042.
He, Y., Jouaux, A., Ford, S. E., Lelong, C., Sourdaine, P., Mathieu, M., et al. (2015). Transcriptome analysis reveals strong and complex antiviral response in a mollusc. Fish Shellfish Immunol. 46, 131-144. doi: 10.1016/j.fsi.2015.05.023.
Helbig, K. J., Lau, D. T. Y., Semendric, L., Harley, H. A. J., and Beard, M. R. (2005). Analysis of ISG expression in chronic hepatitis C identifies viperin as a potential antiviral effector. Hepatology 42, 702-710. doi: 10.1002/hep.20844.
Hwang, J. Y., Park, J. J., Yu, H. J., Hur, Y. B., Arzul, I., Couraleau, Y., et al. (2013). Ostreid herpesvirus 1 infection in farmed Pacific oyster larvae Crassostrea gigas (Thunberg) in Korea. J. Fish Dis. 36, 969-972. doi: 10.1111/jfd.12093.
Jenkins, C., Hick, P., Gabor, M., Spiers, Z., Fell, S., Gu, X., et al. (2013). Identification and characterization of an ostreid herpesvirus-1 microvariant (OsHV-1 u-var) in Crassostrea gigas (Pacific oysters) in Australia. Dis. Aquat. Organ. 105, 109-126. doi: doi.org/10.3354/dao02623.
Kassambara, A. (2020). ggpubr: “ggplot2” Based Publication Ready Plots. Available at: https://cran.r-project.org/web/packages/ggpubr/index.html.
Kassambara, A. (2021). rstatix: Pipe-Friendly Framework for Basic Statistical Tests. Available at: https://cran.r-project.org/web/packages/rstatix/index.html.
Keeling, S. E., Brosnahan, C. L., Williams, R., Gias, E., Hannah, M., Bueno, R., et al. (2014). New Zealand juvenile oyster mortality associated with ostreid herpesvirus 1—an opportunistic longitudinal study. Dis. Aquat. Organ. 109, 231-239. doi: 10.3354/dao02735.
Kirkland, P. D., Hick, P., and Gu, X. (2015). Development of a laboratory model for infectious challenge of Pacific Oysters (Crassostrea gigas) with ostreid herpesvirus type-1. http://frdc.com.au/Archived-Reports/FRDC Projects/2012-052-DLD.pdf.
Labreuche, Y., Lambert, C., Soudant, P., Boulo, V., Huvet, A., and Nicolas, J. (2006). Cellular and molecular hemocyte responses of the Pacific oyster, Crassostrea gigas, following bacterial infection with Vibrio aestuarianus. Microbes Infect. 8, 2715-2724.
Lafferty, K. D., Porter, J. W., and Ford, S. E. (2004). Are diseases increasing in the ocean? Annu. Rev. Ecol. Evol. Syst. 35, 31-54. doi: 10.1146/annurev.ecolsys.35.021103.105704.
Lafont, M., Petton, B., Vergnes, A., Pauletto, M., Segarra, A., Gourbal, B., et al. (2017). Long-lasting antiviral innate immune priming in the Lophotrochozoan Pacific oyster, Crassostrea gigas. Sci. Rep. 7, 1-14. doi: 10.1038/s41598-017-13564-0.
Lafont, M., Vergnes, A., Vidal-dupiol, J., De Lorgeril, J., Gueguen, Y., Haffner, P., et al. (2020). A Sustained Immune Response Supports Long-Term Antiviral Immune Priming in the Pacific Oyster, Crassostrea gigas. Host microbe Biol. 11, 1-17.
Martenot, C., Faury, N., Morga, B., Degremont, L., Lamy, J.-B., Houssin, M., et al. (2019). Exploring First Interactions Between Ostreid Herpesvirus 1 (OsHV-1) and Its Host, Crassostrea gigas: Effects of Specific Antiviral Antibodies and Dextran Sulfate. Front. Microbiol. 10, 1-13. doi: 10.3389/fmich.2019.01128.
Martenot, C., Oden, E., Travaillé, E., Malas, J. P., and Houssin, M. (2010). Comparison of two real-time PCR methods for detection of ostreid herpesvirus 1 in the Pacific oyster Crassostrea gigas. J. Virol. Methods 170, 86-89. doi: 10.1016/j.jviromet.2010.09.003.
Milutinović, B., and Kurtz, J. (2016). Immune memory in invertebrates. Semin. Immunol. 28, 328-342. doi: 10.1016/j.smim.2016.05.004.
Moreira, R., Romero, A., Rey-Campos, M., Pereiro, P., Rosani, U., Novoa, B., et al. (2020). Stimulation of Mytilus galloprovincialis Hemocytes With Different Immune Challenges Induces Differential Transcriptomic, miRNomic, and Functional Responses. Front. Immunol. 11. doi: 10.3389/fimmu.2020.606102.
Morga, B., Arzul, I., Chollet, B., and Renault, T. (2009). Infection with the protozoan parasite Bonamia ostreae modifies in vitro haemocyte activities of flat oyster Ostrea edulis. Fish Shellfish Immunol. 26, 836-842. doi: 10.1016/j.fsi.2009.03.018.
Morga, B., Faury, N., Guesdon, S., Chollet, B., and Renault, T. (2017). Haemocytes from Crassostrea gigas and OsHV-1: A promising in vitro system to study host/virus interactions. J. Invertebr. Pathol. 150, 45-53. doi: 10.1016/j.jip.2017.09.007.
Nasr, N., Maddocks, S., Turville, S. G., Harman, A. N., Woolger, N., Helbig, K. J., et al. (2012). HIV-1 infection of human macrophages directly induces viperin which inhibits viral production. Blood 120, 778-788. doi: 10.1182/blood-2012-01-407395.
Netea, M. G., Domínguez-Andrés, J., Barreiro, L. B., Chavaki, T., Divangahi, M., Fuchs, E., et al. (2020). Defining trained immunity and its role in health and disease. Nat. Rev. Immunol. 20. doi: 10.1038/s41577-020-0285-6.
Netea, M. G., Quintin, J., and Van Der Meer, J. W. M. (2011). Trained immunity: A memory for innate host defense. Cell Host Microbe 9, 355-361. doi: 10.1016/j.chom.2011.04.006.
Pauletto, M., Milan, M., Moreira, R., Novoa, B., Figueras, A., Babbucci, M., et al. (2014). Deep transcriptome sequencing of Pecten maximus hemocytes: A genomic resource for bivalve immunology. Fish Shellfish Immunol. 37, 154-165. doi: 10.1016/j.fsi.2014.01.017.
Pernet, F., Lupo, C., Bacher, C., and Whittington, R. J. (2016). Infectious diseases in oyster aquaculture require a new integrated approach. Philos. Trans. R. Soc. B Biol. Sci. 371. doi: 10.1098/rstb.2015.0213.
Picot, S., Faury, N., Pelletier, C., Arzul, I., Chollet, B., Dégremont, L., et al. (2022). Monitoring Autophagy at Cellular and Molecular Level in Crassostrea gigas During an Experimental Ostreid Herpesvirus 1 (OsHV-1) Infection. Front. Cell. Infect. Microbiol. 12, 1-14. doi: 10.3389/fcimb.2022.858311.
Rolton, A., Delisle, L., Berry, J., Venter, L., Charles, S., Adams, S., et al. (2020). Flow cytometric characterization of hemocytes of the flat oyster, Ostrea chilensis. Fish Shellfish Immunol. 97, 411-420. doi: 10.1016/j.fsi.2019.12.071.
Rosani, U., Varotto, L., Domeneghetti, S., Arcangeli, G., Pallavicini, A., and Venier, P. (2015). Dual analysis of host and pathogen transcriptomes in ostreid herpesvirus 1-positive Crassostrea gigas. Environ. Microbiol. 17, 4200-4212. doi: 10.1111/1462-2920.12706.
Saeed, A. I., Sharov, V., White, J., Li, J., Liang, W., Bhagabati, N., et al. (2003). TM4: A free, open-source system for microarray data management and analysis. Biotechniques 34, 374-378. doi: 10.2144/03342mt01.
Sanders, B., Koldijk, M., and Schuitemaker, H. (2015). “Inactivated viral vaccines,” in Vaccine Analysis: Strategies, Principles, and Control (Springer), 45-80. doi: 10.1007/978-3-662-45024-6.
Segarra, A., Baillon, L., Faury, N., Tourbiez, D., and Renault, T. (2016). Detection and distribution of ostreid herpesvirus 1 in experimentally infected Pacific oyster spat. J. Invertebr. Pathol. 133, 59-65. doi: 10.1016/j.jip.2015.11.013.
Segarra, A., Faury, N., Pépin, J. F., and Renault, T. (2014). Transcriptomic study of 39 ostreid herpesvirus 1 genes during an experimental infection. J. Invertebr. Pathol. 119, 5-11. doi: 10.1016/j.jip.2014.03.002.
Sheehan, G., Farrell, G., and Kavanagh, K. (2020). Immune priming: the secret weapon of the insect world. Virulence. doi: 10.1080/21505594.2020.1731137.
The International Bank for Reconstruction and Development (2014). Reducing disease risk in aquaculture world bank report number 88257-GLB. doi: 10.13140/RG.2.1.4525.5529.
Virol, A., and Feng, S. (2017). Recent progress in the development of white spot syndrome virus vaccines for protecting shrimp against viral infection. Arch. Virol. doi: 10.1007/s00705-017-3450-x.
Wang, L., Song, X., and Song, L. (2018). The oyster immunity. Dev. Comp. Immunol. 80, 99-118. doi: 10.1016/j.dci.2017.05.025.
Wiethoelter, A. K., Beltrán-Alcrudo, D., Kock, R., and Mor, S. M. (2015). Global trends in infectious diseases at the wildlife-livestock interface. Proc. Natl. Acad. Sci. U.S.A. 112, 9662-9667. doi: 10.1073/pnas.1422741112.
Witteveldt, J., Cifuentes, C. C., Vlak, J. M., and Hulten, C. W. Van (2004). Protection of Penaeus monodon against White Spot Syndrome Virus by Oral Vaccination. J Virol. 78, 2057-2061. doi: 10.1128/JVI.78.4.2057.
Yang, W., Tran, N. T., Zhu, C., Zhang, M., Yao, D., Aweya, J. J., et al. (2020). Enhanced immune responses and protection against the secondary infection in mud crab (Scylla paramamosain) primed with formalin-killed Vibrio parahemolyticus. Aquaculture 529, 735671. doi: 10.1016/j.aquaculture.2020.735671.
Yao, T., Lu, J., Bai, C., Xie, Z., and Ye, L. (2021). The Enhanced Immune Protection in Small Abalone Haliotis diversicolor Against a Secondary Infection with Vibrio harveyi. Front. Immunol. 12, 1-13. doi: 10.3389/fimmu.2021.685896.
Yu, J., Liu, Y., Huang, B., Li, C., Wang, D., Yao, M., et al. (2021). Characterization of host cell potential proteins interacting with OsHV-1 membrane proteins. Viruses 13, 1-13. doi: 10.3390/v13122518.
Zhang, T., Qiu, L., Sun, Z., Wang, L., Zhou, Z., Liu, R., et al. (2014). The specifically enhanced cellular immune responses in Pacific oyster (Crassostrea gigas) against secondary challenge with Vibrio splendidus. Dev. Comp. Immunol. 45, 141-150. doi: 10.1016/j.dci.2014.02.015.
Zhu, F., Qian, X., and Ma, X. (2018). Comparative transcriptomic analysis of crab hemocytes in response to white spot syndrome virus or Vibrio alginolyticus infection. Fish Shellfish Immunol. 80, 165-179. doi: 10.1016/j.fsi.2018.06.003.

1) A method of potentiating the innate immune response of a mollusk against a pathogen comprising contacting the mollusk with a pathogen-derived antigen where the pathogen-derived antigen has been inactivated by contacting with an ethylenimine.

2) The method of claim 1 where the contacting is in a hatchery and the mollusk is to be transferred to a farm.

3) The method of claim 2 where the mollusk is Crassostrea gigas and the pathogen is Ostreid herpes virus-1.

4) The method of claim 2 where the mollusk is Ostrea edulis and the pathogen is Bonamia ostreae.

5) The method of claim 2 where the mollusk is Perna canaliculus and the pathogen is Perkinsus olseni.

6) The method of claim 2 where the mollusk is one or more species of mussel or oyster and the pathogen is Vibrio sp.

7) A composition for use in a method of potentiating the innate immune response of a mollusk comprising an inactivated pathogen-derived antigen where the pathogen-derived antigen has been inactivated by contacting with binary ethylenimine (BEI).

8) The composition of claim 7 comprising an inactivated pathogen-derived antigen dispersed in sterile seawater.

9) The composition of claim 7 where the pathogen is Ostreid herpes virus-1.

10) The composition of claim 7 where the pathogen is Bonamia ostreae.

11) The composition of claim 7 where the pathogen is Perkinsus olseni.

12) The composition of claim 7 where the pathogen is Vibrio sp.

13) A method of preparing a composition for use in a method of potentiating the innate immune response of a mollusk comprising inactivating a pathogen-derived antigen by contacting with an ethylenimine.

14) The method of claim 13 where the ethylenimine is binary ethylenimine (BEI).

15) The method of claim 14 where the pathogen-derived antigen is virions of Ostreid herpes virus-1.
